(19) United States
(12) Patent Application Publication
(10) Pub. No.: US 2006/0057689 A1
(43) Pub. Date: Mar. 16, 2006

(54) Method for producing c4-c12 fatty acids

(76) Inventors: Ralf Otto, Oggelshausen (DE); Georg Fieg, Mettmann (DE); Sabine Both, Duesseldorf (DE); Ulrich Schoerken, Duesseldorf (DE); Levent Yueksel, Duesseldorf (DE); Ingomar Mrozek, Duesseldorf (DE); Carolin Meyer, Duesseldorf (DE); Norbert Klein, Mettmann (DE); Albrecht Weiss, Langenfeld (DE)

Correspondence Address: COGNIS CORPORATION;PATENT DEPARTMENT, 300 BROOKSIDE AVENUE, AMBLER, PA, 19002, US

(21) Appl. No.: 10/513,812

(22) PCT Filed: Apr. 29, 2003
(86) PCT No.: PCT/EP03/04440

§ 371 Date: Jul. 15, 2005

(30) Foreign Application Priority Data
May 8, 2002 (DE) 102 20 525.6

(51) Int. Cl. C12P 7/64; C12P 7/40

(52) U.S. Cl. 435/134; 435/136

(57) ABSTRACT

Processes for preparing fatty acids are described wherein a C₄-C₁₂ fatty acid methyl ester is subjected to hydrolysis in the presence of an enzyme to form an organic phase comprising a C₄-C₁₂ fatty acid and an aqueous phase comprising methanol, wherein at least a portion of the methanol is continuously removed; and the organic phase is subsequently separated from the aqueous phase, and optionally, where the organic phase further comprises an unhydrolyzed portion of the C₄-C₁₂ fatty acid methyl ester, the unhydrolyzed portion of the C₄-C₁₂ fatty acid methyl ester is separated from the C₄-C₁₂ fatty acid.

### FIELD OF THE INVENTION

The invention is within the field of oleochemical raw materials and relates to a biotechnological method of preparing short-chain fatty acids from the corresponding methyl esters.

### PRIOR ART

In oleochemistry, fatty acid methyl esters of differing chain length distribution are produced. When longer-chain fatty acid methyl esters are separated off by distillation, what are termed first runnings fatty acid methyl esters are produced which are highly differing mixtures of C₄- to C₁₂-methyl esters and which are frequently directly used in further transesterification reactions. The resultant derivatives are, however, owing to the impure raw material, of poor quality. Alternatively, therefore, the fatty acid methyl esters are first cleaved and the released fatty acids are then esterified. Chemical hydrolysis is performed in the presence of acid catalysts, for example alkylbenzenesulfonic acids, disclosed by international application WO 94/14743. In the method, therefore, sulfuric acid is formed which leads in the plants to great corrosion and the products are contaminated by high metal contents. In addition, the yield of these methods is not yet optimum. A further problem is environmentally compatible disposal of the catalysts.

An object of the present invention was thus to provide an improved method of preparing short-chain fatty acids from their methyl esters, which method reliably avoids said disadvantages of the prior art. In particular, the fatty acids should be obtained in high purity and high yields and the method should operate under mild conditions.

### DESCRIPTION OF THE INVENTION

The invention relates to a method of preparing C₄-C₁₂ fatty acids in which
(a) C₄-C₁₂ fatty acid methyl esters are completely or partially hydrolyzed in one stage in the presence of enzymes with water and continuous removal of methanol,
(b) the hydrolysate is separated into an organic phase and an aqueous/alcoholic phase,
(c) and the organic phase comprising fatty acids and (in the case of partial hydrolysis) fatty acid methyl esters is freed from unreacted fatty acid methyl esters.

Surprisingly it has been found that enzymatic hydrolysis with continuous removal of methanol leads to fatty acids which are free from unwanted byproducts. High yields are achieved, the method operates under mild conditions and uses catalysts which meet all requirements of environment compatibility.

If, during the hydrolysis method, the methanol is removed continuously directly from the hydrolysis reactor, a much more rapid reaction is achieved in a single-stage method.

Hydrolysis

The fatty acid methyl esters are preferably hydrolyzed at mild temperatures in the range from 20 to 80° C., preferably from 30 to 70° C., and particularly preferably from 35 to 60° C., with continuous removal of methanol under vacuum, the preferred temperature being preset by the activity optimum of the enzymes used. Usually, the lipases and/or esterases are used in free or immobilized form. Typical examples of suitable enzymes, but which is not to be limiting, are lipases and/or esterases of microorganisms selected from the group consisting of Alcaligenes, Aspergillus niger, Candida antarctica A, Candida antarctica B, Candida cylindracea, Chromobacterium viscosum, Rhizomucor miehei, Penicilium camenberti, Penicilium roqueforti, Porcine pancreas, Pseudomonas cepacia, Pseudomonas fluorescens, Rhizopus javanicus, Rhizopus oryzae, Thermomyces lanugenosus (see Example 1). Preference is given to lipases and esterases from the organisms Alcaligenes, Candida, Chromobacterium, Rhizomucor, Pseudomonas, Rhizopus and Thermomyces.

The enzymes are generally used as dilute suspensions or aqueous concentrates. The lipases/esterases can also be used in immobilized form on support material and reused in repeated batches.

A suitable hydrolysis method is a batch procedure in which a constant water content is set, usually in the range from 30-70% by weight in the reactor, via resupply of water. Usually, the reaction is carried out at a temperature of 30-50° C. and below 100 mbar, preferably 50 to 70 mbar (Examples 2, 3, 4 and 6).

Also suitable is a hydrolysis method implementing a batch procedure in which the water is continuously fed in and methanol/water continuously stripped off. Usually, the water content in the reactor in this procedure is low (0-20% by weight). The reaction is usually carried out at a temperature of 50-70° C. and below 100 mbar, preferably 50 to 70 mbar (Examples 7 and 8).

Methods in which the methanol removal is separated from the hydrolysis reaction in space and/or time operate markedly worse. Such a disadvantageous process is described in JP05317063.

Examples of less suitable methods are methanol removal in a separate reaction vessel (Example 9) and methanol removal via a dephlegmator or, for example, a falling-film evaporator (Example 10), in which the organic phase and aqueous phase are continuously recycled to the hydrolysis reactor. One example of separation of methanol removal and hydrolysis in time is described in Example 11 and 12. A multistage method according to this plan leads to lower yields of short-chain fatty acids.

Workup

Subsequently to the hydrolysis, the aqueous/alcoholic phase is separated from the organic phase and the latter is worked up, that is to say unreacted methyl ester is removed from the product of value.

Depending on the hydrolysis time, differing cleavage rates are obtained. The reaction can be terminated early, for example already in the range of a conversion rate of 60% by weight, so that the fatty acids and fatty acid methyl esters must subsequently be separated by distillation. However, it can also be terminated not until greater than 90% by weight, preferably greater than 95% by weight, or even continued up to 99% by weight, so that as in the latter case no further subsequent separation is necessary.

The unreacted methyl ester is preferably removed in a distillation column containing packed internals, in which case it has proved to be advantageous to supply the feed between the enrichment part and the stripping part of the column. At temperatures in the range from 70 to 100° C. and at a reduced pressure of from 10 to 50 mbar, the methyl esters are taken off at the top of the column and can be recirculated to the reaction. Shorter-chain fatty acids and low-boiling impurities can be drawn off via the pump and pass into the exhaust air, for which reason a downstream condensation is advisable. The resultant fatty acids have a purity of at least 95% by weight.

### EXAMPLES

### Example 1

Selection of Suitable Lipases for the Hydrolysis of Short-Chain Fatty Acid Methyl Esters

15 batches each having 4 g of C8 fatty acid methyl ester (methyl caprylate) and 6 g of water in a sealable reaction vessel are provided with stirrer bars and stirred on a multiple stirrer plate in parallel at room temperature. To the batches, in each case commercially available lipases or esterases are added in accordance with the table given below. After a reaction time of 2 h and 24 h, samples are taken in each case. The organic phase containing fatty acid methyl ester and enzymatically hydrolyzed fatty acid is separated and analyzed.

TABLE 1
Lipases and esterases used

| Enzyme | Organism | Manufacturer | mg/batch |
|---|---|---|---|
| Chirazym L-10 | Alcaligenes sp. | Roche | 40 |
| Lipase A | Aspergillus niger | Amano | 40 |
| Novozym 868 | Candida antarctica A | Novozymes | 40 |
| Novozym 525 | Candida antarctica B | Novozymes | 40 |
| Lipomod 34 | Candida cylindracea | Biocatalysts | 40 |
| Lipase LP | Chromobacterium viscosum | Asahi Kasei | 4 |
| Novozym 388 | Rhizomucor miehei | Novozymes | 40 |
| Lipase G | Penicilium camenberti | Amano | 40 |
| Lipase R | Penicilium roqueforti | Amano | 40 |
| Lipase L115P | Porcine pancreas | Biocatalysts | 40 |
| Lipase PS | Pseudomonas cepacia | Amano | 40 |
| Lipase AK | Pseudomonas fluorescens | Amano | 40 |
| Lipomod 36 P | Rhizopus javanicus | Biocatalysts | 40 |
| Lipase F-AP 15 | Rhizopus oryzae | Amano | 40 |
| Lipolase | Thermomyces lanugenosus | Novozymes | 40 |
| TI 100 | | | |

The conversion rate of the reaction was studied via determination of the acid number.

TABLE 2
Conversion rate using differing enzymes after a reaction
time of 2 and 24 h

| | Conversion rate [2 h | Conversion rate |
|---|---|---|
| Enzyme | reaction] | [24 h reaction] |
| Chirazym L-10 | 18.4% | 33.7% |
| Lipase A | 0.9% | 8.8% |
| Novozym 868 | 2.4% | 8.3% |
| Novozym 525 | 27.3% | 34.7% |
| Lipomod 34 | 22.9% | 31.4% |
| Lipase LP | 23.9% | 36.1% |
| Novozym 388 I | 18.5% | 26.1% |
| Lipase G | 1.6% | 19.7% |
| Lipase R | 0.9% | 2.0% |
| Lipase L115P | 4.0% | 12.9% |
| Lipase PS | 16.3% | 27.4% |
| Lipase AK | 11.6% | 28.3% |
| Lipomod 36 P | 8.5% | 24.5% |
| Lipase F-AP 15 | 12.7% | 12.3% |
| Lipolase TI 100 | 18.4% | 24.1% |

All of the lipases and esterases tested had a hydrolysis activity for short-chain fatty acid methyl esters. After screening, those which are to be preferred are lipases and esterases from the organisms Alcaligenes, Candida, Chromobacterium, Rhizomucor, Pseudomonas, Rhiozopus and Thermomyces.

### Example 2

Cleavage of Short-Chain Fatty Acid Methyl Esters with Continuous Removal of MeOH

2800 g of water, 1200 g of fatty acid methyl ester and 40 ml of Lipolase (Thermomyces lipase, Novozymes) are charged into a thermostatable jacketed reactor having an attached Sulzer column. The reaction is carried out at a reactor temperature of 35° C. and a vacuum of 60 mbar. The reflux/take off ratio at the column head is set to 12:2. The stirrer speed is set to 300 rpm.

After 5 h, 40 ml of Lipolase and 500 ml of water are added.

The conversion rate of the reaction was studied via determination of the acid number.

TABLE 3
Conversion rate with continuous removal of methanol
after differing reaction times

| | Conversion rate according to acid |
|---|---|
| Reaction time [h] | number [%] |
| 0 | 0 |
| 2.5 | 42 |
| 23 | 70.6 |
| 30 | 75.5 |
| 44 | 82.6 |
| 48 | 84 |
| 52 | 86.5 |
| 68 | 91.6 |
| 92 | 94 |

In the distillate, the amount of distillate present and thus removed from the reaction equilibrium is determined.

TABLE 4
Conversion rate with continuous removal of methanol - amount
of distillate removed from reaction equilibrium after differing
reaction times

| Reaction time [h] | Amount of distillate [g] | Methanol content [%] |
|---|---|---|
| 0-4.5 | 10 | |
| 4.5-44 | 80 | 40 |
| 44 | 5 | 25 |
| 44-68 | 190 | 14 |
| 68 | 3 | 5 |
| 68-92 | 160 | 4 |
| 92 | 3 | 2 |

In the aqueous phase of the reaction batch, <0.2% methanol was found after termination of the reaction.

### Example 3

Cleavage of Short-Chain Fatty Acid Methyl Esters with Continuous Removal of MeOH

2800 g of water, 1200 g of fatty acid methyl ester and 40 ml of Novozym (Candida antarctica B lipase, Novozymes) are charged into a thermostatable jacketed reactor having an attached Sulzer column. The reaction is carried out at a reactor temperature of 35° C. and a vacuum of 60 mbar. The reflux/take off ratio at the column head is set to 12:2. The stirrer speed is set to 300 rpm.

After 24 h, 300 g of water are added.

The conversion of the reaction was studied via determination of the acid number.

TABLE 5
Conversion rate with continuous removal of methanol
after differing reaction times

| | Conversion rate according to acid |
|---|---|
| Reaction time [h] | number [%] |
| 0 | 0 |
| 2 | 47 |
| 3 | 50.1 |
| 4 | 52.7 |
| 6 | 58.1 |
| 21 | 82.5 |
| 27 | 87.8 |
| 45 | 95.6 |

### Example 4

Cleavage of Short-Chain Fatty Acid Methyl Esters Using Immobilized Lipase with Continuous Removal of MeOH

350 g of water, 350 g of fatty acid methyl ester and 35 g of immobilized Novozym (Candida antarctica B lipase, Novozymes, adsorbed to a polypropylene support, enzyme loading 200 mg of technical grade liquid preparation per g of support) are charged into a heated flask having an attached dephlegmator. The reaction is carried out at a reactor temperature of 35° C. and a vacuum of 60 mbar. Water is continuously added to the batch at about 0.75 ml/min, so that the reactor volume remains constant over the course of time.

The conversion rate of the reaction was studied via determination of the acid number.

TABLE 6
Conversion rate with continuous removal of methanol using
immobilized lipase after differing reaction times

| | Conversion rate according to acid |
|---|---|
| Reaction time [h] | number [%] |
| 0 | 0 |
| 1 | 44.0 |
| 2 | 52.7 |
| 3 | 61.6 |
| 4 | 66.8 |
| 5 | 71.4 |
| 24 | 94.8 |

### Example 5

Study of the Stability of Immobilized Lipase in the Cleavage of Short-Chain Fatty Acid Methyl Esters

For the stability study, Candida antarctica B lipase (Novozym 525, Novozymes) is used which had previously been adsorbed to polypropylene supports. Studies are carried out at room temperature, 50° C., 60° C. and 70° C. For this, the immobilized lipases are stirred in a mixture of short-chain fatty acid methyl esters (mixture of C6-C10 fatty acids, 50% by weight) and water (50% by weight) until a reaction equilibrium is established. At intervals (see results in table), the immobilized enzyme is filtered off and admixed with fresh fatty acid methyl ester and water. The respective hydrolysis rate is determined.

The conversion rate of the reaction was studied via determination of the acid number after a reaction time of 4 h.

TABLE 7
Conversion rate at differing temperatures using immobilized lipase
stored for differing times

| | | Conversion | Conversion | Conversion | Conversion |
|---|---|---|---|---|---|
| | | rate | rate | rate | rate |
| Storage time | | (RT) | (50° C.) | (60° C.) | (70° C.) |
| 1st | week | 28.6% | 32.7% | 33.0% | 34% |
| 2nd | week | 28.8% | 33.4% | 33.5% | 7.4% |
| 3rd | week | 29.7% | 33.2% | 34.1% | 4.5% |
| 6th | week | 25.0% | 29.1% | 30.9% | 4.9% |
| 13th | week | 26.1% | 15.2% | 13.2% | 4.5% |
| 16th | week | 26.5% | 7.2% | 6.5% | 2.5% |

The half-life of the enzyme at 50° C. is about 12 weeks, at 60° C. about 10 weeks, at 70° C. about 1 week, and at room temperature is over 16 weeks.

### Example 6

Hydrolysis of Short-Chain Fatty Acid Methyl Esters on a Pilot Scale Using Immobilized Lipase in Repeated Batches

25 kg of water, 20 kg of fatty acid methyl ester Edenor Me C 6-10 and 2.5 kg of immobilized Novozym (Candida antarctica B lipase, Novozymes, adsorbed on polypropylene supports, enzyme loading 200 mg of technical grade liquid preparation per g of support) are charged into a thermostatable jacketed reactor having an attached total condenser and complete distillate-take off. The reaction is carried out at a reactor interior temperature of 45° C. and a vacuum of 60 mbar. The stirrer speed is set to 150 rpm. Since under said conditions a methanol/water distillate is produced, water must continuously be added to the batch so that the volume in the reactor remains constant over the course of time. After completion of the reaction, the reaction mixture is drained off from the vessel, the immobilized enzyme being retained in the reactor via a built-in screen.

The conversion of each batch is compared after 12 hours. The conversion rate of the reaction was studied via determination of the acid number.

TABLE 8
Conversion rate with continuous removal of methanol
on a pilot scale

| Batch | Conversion rate [%] |
|---|---|
| 1 | 75 |
| 2 | 73 |
| 3 | 76 |
| 10 | 75 |
| 20 | 75 |
| 30 | 75 |
| 40 | 75 |

The immobilized enzyme, even after 40 batches, under the parameters chosen, showed no loss of activity, which was correlated with the conversion rate.

### Example 7

Continuous Hydrolysis of Short-Chain Fatty Acid Methyl Esters, Stripping off Water and Methanol

200 g of fatty acid methyl ester, 20 g of water and 10 g of Candida antarctica B lipase immobilized on polypropylene are charged into a heatable flask. The reaction is carried out using an attached distillation bridge at 60 mbar and a temperature of 60° C. Water is continuously pumped into the flask at a flow rate of 0.25 ml/min. In a second batch, water is pumped into the flask at a flow rate of 0.5 ml/min. The water content in the flask is less than 20% over the entire reaction period.

The conversion rate of the reaction was studied via determination of the acid number.

TABLE 9
Conversion rate with stripping off of water and methanol
after differing reaction times

| | Conversion rate [%] | Conversion rate [%] |
|---|---|---|
| Reaction time [h] | flow rate 0.25 ml/min | flow rate 0.5 ml/min |
| 0 | 0 | 0 |
| 2 | 37.1 | |
| 4 | | 61.5 |
| 16 | 83.9 | |
| 23 | | 96.0 |
| 24 | 90.4 | |
| 29.5 | | 96.9 |
| 52 | 95.5 | |

### Example 8

Continuous Hydrolysis of Short-Chain Fatty Acid Methyl Esters, Stripping Off Water and Methanol

200 g of fatty acid methyl ester, 20 g of water and 10 g of Candida antarctica B lipase immobilized on polypropylene are charged into a heatable flask. The reaction is carried out using an attached distillation bridge at 60 mbar and a temperature of 70° C. Water is continuously pumped into the flask at a flow rate of 0.75 ml/min. The water content in the flask is less than 20% over the entire reaction period.

The conversion rate of the reaction was studied via determination of the acid number.

TABLE 10
Conversion rate with stripping off of water and methanol
after differing reaction times

| Reaction time [h] | Conversion rate [%] |
|---|---|
| 0 | 0 |
| 2 | 51.0 |
| 4 | 81.7 |
| 13 | 96.4 |

### Example 9

Continuous Cleavage of Short-Chain Fatty Acid Methyl Esters with Separation of the Cleavage Process and Methanol Removal

350 ml of fatty acid methyl ester, 350 ml of water and 35 g of immobilized Candida antarctica B lipase are placed in a heatable flask. The hydrolysis reaction is carried out at 35° C. The reaction mixture is continuously pumped into a second flask which is heated to 120° C. At a vacuum of 740 mbar, methanol is continuously removed in this flask. The reaction mixture from flask 2 is continuously pumped back to the reaction flask at the same flow rate. Water is added to the reaction flask so as to maintain a constant reaction volume.

Result:

The conversion rate of the reaction was studied via determination of the acid number.

TABLE 11
Conversion rate with separation of cleavage process and methanol
removal after differing reaction times

| Reaction time [h] | Conversion rate [%] |
|---|---|
| 0 | 0 |
| 1 | 32.0 |
| 2 | 36.0 |
| 3 | 37.2 |
| 4 | 38.5 |
| 5 | 39.2 |
| 6 | 39.8 |
| 7 | 40.5 |
| 24 | 47.3 |

The hydrolysis reaction is markedly slower than in the case of continuous methanol removal directly from the reaction flask.

### Example 10

Continuous Cleavage of Short-Chain Fatty Acid Methyl Esters with Separation of Cleavage Process and Methanol Removal

350 ml of fatty acid methyl ester, 350 ml of water and 35 g of immobilized Candida antarctica B lipase are placed in a heatable flask. The hydrolysis reaction is carried out at 45° C. The reaction mixture is pumped continuously through a dephlegmator which is heated to 110° C. At a vacuum of 740 mbar, methanol is removed continuously and the remaining reaction mixture drips back into the reaction flask. Water is added to keep the reaction volume constant.

The conversion rate of the reaction was studied via determination of the acid number.

TABLE 12
Conversion rate with separation of cleavage process and methanol
removal after differing reaction times

| Reaction time [h] | Conversion rate [%] |
|---|---|
| 0 | 0 |
| 1 | 32.7 |
| 2 | 38.4 |
| 3 | 47.2 |
| 4 | 51.6 |
| 5 | 55.2 |
| 6 | 55.9 |
| 24 | 74.8 |

The hydrolysis reaction is markedly slower than in the case of continuous methanol removal directly from the reaction flask.

### Example 11

Multistage Cleavage of Short-Chain Fatty Acid Methyl Esters with Exchange of the Water Phase without Continuous Methanol Removal

7.5 g of fatty acid methyl ester, 12.5 g of water and 0.1 g of Lipolase (Thermomyces lipase, Novozymes) are brought to reaction at room temperature in a stirred vessel. After 18 h, 26 h and 41 h the water phase in each case is removed from the organic phase by separation. In each case 12.5 g of water and 0.1 g of Lipolase are added after each phase exchange.

The conversion rate of the reaction was studied via determination of the acid number.

TABLE 13
Conversion rate with multistage cleavage after differing reaction times

| Reaction time [h] | Conversion rate [%] |
|---|---|
| 0 | 0 |
| 18 | 38.8 |
| 26 | 55.9 |
| 41 | 70.2 |
| 60 | 81.8 |

### Example 12

Two-Stage Cleavage of Short-Chain Fatty Acid Methyl Esters

540 g of a first runnings C₈ fatty acid methyl ester were placed in a stirred apparatus having a capacity of 3 l, 1260 g of water and 16.2 ml of Lipolase concentrate were added and the mixture was stirred at 37° C. with a speed of 300 rpm. The hydrolysis was followed by sampling. After 16 h the hydrolysis was interrupted, the resultant first hydrolysate was transferred to a centrifuge and separated into an aqueous/alcoholic phase (containing water, methanol and enzymes) and an organic phase which contains the fatty acid formed and the unreacted methyl ester. The organic phase was recirculated to the reactor, and 1260 g of water and 16.2 ml of fresh enzyme were added. The mixture was then subjected to a second hydrolysis, again at 37° C. Here also the progress of the reaction was followed by sampling. The results are summarized in Table 14.

TABLE 14
Conversion rate of hydrolysis of first runnings fatty acid methyl esters
(figures in % by weight)

| Hydrolysis | 1st hydrolysis | | 2nd hydrolysis | |
|---|---|---|---|---|
| time [h] | Methyl ester | Fatty acid | Methyl ester | Fatty acid |
| 0.25 | 80.2 | 18.0 | | |
| 0.5 | | | 46.2 | 51.8 |
| 0.75 | 61.1 | 36.9 | | |
| 1.0 | 57.6 | 40.4 | | |
| 1.5 | 49.6 | 48.3 | 42.5 | 55.5 |
| 2.5 | | | 40.5 | 57.5 |
| 4.5 | | | 37.2 | 60.8 |
| 16 | 45.0 | 53.0 | | |
| 20 | | | 30.8 | 67.1 |

The second hydrolysate which contained 67.1% by weight fatty acid and 30.8% by weight of unreacted methyl ester was again separated by centrifugation into an aqueous/alcoholic phase and an organic phase. The latter was passed into a rectification column, between the enrichment part and the stripping part, equipped with packed internals and distilled at 85° C. and 20 mbar. After 6 h, while the shorter-chain and low-boiling impurities were withdrawn via a pump, a C₈ fatty acid was obtained at a purity of greater than 95% by weight.

### Example 13

Comparison of Various Enzymatic Methods for Hydrolyzing Short-Chain Fatty Acid Methyl Esters

Comparison of methods from Examples 4, 7, 9, 10, 11.

Example 4 describes a hydrolysis method with continuous methanol removal at a constant water content in the reactor.

Example 7 describes a hydrolysis method with continuous methanol removal in which water is continuously stripped from the reaction vessel. The water content of the reaction vessel is low here.

Example 9 describes a hydrolysis method with continuous methanol removal in which the methanol removal and the hydrolysis reaction are separated in space.

Example 10 describes an alternative hydrolysis method with continuous methanol removal in which the methanol removal and the hydrolysis reaction are separated in space.

Example 11 describes a hydrolysis method without continuous methanol take off under vacuum, in which methanol is withdrawn from the equilibrium via separation of the aqueous phase.

TABLE 15
Comparison of different methods

| | Con- | Con- | | | |
|---|---|---|---|---|---|
| | version | version | Conversion | Conversion | Conversion |
| Time | rate [%] | rate [%] | rate [%] | rate [%] | rate [%] |
| [h] | Example 4 | Example 7 | Example 9 | Example 10 | Example 11 |
| 0 | 0 | 0 | 0 | 0 | 0 |
| 1 | 44.0 | | 32.0 | 32.7 | |
| 2 | 52.7 | 37.1 | 36.0 | 38.4 | |
| 5 | 71.4 | | 39.2 | 55.2 | |
| 16 | | 83.9 | | | |
| 18 | | | | | 38.8 |
| 24 | 94.8 | 90.4 | 47.3 | 74.8 | |
| 26 | | | | | 55.9 |
| 41 | | | | | 70.2 |
| 53 | | 95.5 | | | |
| 60 | | | | | 81.8 |

Comparison of the methods clearly shows that methanol removal directly from the reaction batch gives the best conversion rates. Spatial separation of the hydrolysis and methanol removal with continuous methanol take off in a separate vessel or separation in time of hydrolysis and methanol removal, via, for example, phase separation, does not lead to satisfactory results.

1-7. (canceled)

8. A process comprising:
(a) subjecting a C₄-C₁₂ fatty acid methyl ester to hydrolysis in the presence of an enzyme to form an organic phase comprising a C₄-C₁₂ fatty acid and an aqueous phase comprising methanol, wherein at least a portion of the methanol is continuously removed; and
(b) separating the organic phase and the aqueous phase.

9. The process according to claim 8, wherein the organic phase further comprises an unhydrolyzed portion of the C₄-C₂ fatty acid methyl ester, and the C₄-C₁₂ fatty acid is separated from the unhydrolyzed portion of the C₄-C₁₂ fatty acid methyl ester.

10. The process according to claim 8, wherein the hydrolysis is carried out at a temperature of from 20 to 80° C.

11. The process according to claim 9, wherein the hydrolysis is carried out at a temperature of from 20 to 80° C.

12. The process according to claim 8, wherein the methanol removal is carried out under at least a partial vacuum.

13. The process according to claim 9, wherein the methanol removal is carried out under at least a partial vacuum.

14. The process according to claim 10, wherein the methanol removal is carried out under at least a partial vacuum.

15. The process according to claim 8, wherein the hydrolysis is carried out in a reaction zone and the methanol is removed directly from the reaction zone.

16. The process according to claim 10, wherein the hydrolysis is carried out in a reaction zone and the methanol is removed directly from the reaction zone.

17. The process according to claim 12, wherein the hydrolysis is carried out in a reaction zone and the methanol is removed directly from the reaction zone.

18. The process according to claim 8, wherein water is present during hydrolysis in an amount of from 30 to 70% by weight.

19. The process according to claim 8, wherein water is present during hydrolysis in an amount of up to 20% by weight.

20. The process according to claim 8, wherein the enzyme comprises a microorganism selected from the group consisting of Alcaligenes, Candida, Chromobacterium, Rhizomucor, Pseudomonas, Rhizopus and Thermomyces.
